(19) United States
(12) Patent Application Publication
(10) Pub. No.: US 2006/0122488 A1
(43) Pub. Date: Jun. 8, 2006

(54) URODYNAMIC DIAGNOSTIC METHOD AND SYSTEM

(76) Inventor: Abdol-Mohammad Kajbafzadeh, Tehran (IR)

Correspondence Address: MORRIS MANNING & MARTIN LLP, 1600 ATLANTA FINANCIAL CENTER, 3343 PEACHTREE ROAD, NE, ATLANTA, GA, 30326-1044, US

(21) Appl. No.: 10/904,610

(22) Filed: Nov. 18, 2004

(51) Int. Cl. A61B 5/05; A61B 5/00

(52) U.S. Cl. 600/411; 600/561

(57) ABSTRACT

A method for obtaining information about the function of the lower urinary tract of a patient is disclosed. The method comprising the steps of: measuring the pressure within the lower urinary tract of the patient; and simultaneously imaging the lower urinary tract and carrying out a dynamic pelvic floor study of the patient with an MRI machine. A system is also disclosed. The system is for use with an MRI machine, for obtaining information as to the function of the lower urinary tract of a patient. The system comprises a urodynamic machine for measuring the pressure within the lower urinary tract of the patient while the lower urinary tract is imaged by the MRI machine.

### FIELD OF THE INVENTION

The present invention relates to the field of medical diagnostics, and more particularly, to a method for obtaining information about the lower urinary tract functionality of a patient. A system, for use with a MRI machine and for obtaining information about the functionality of the lower urinary tract of a patient, is also disclosed.

### BACKGROUND OF THE INVENTION

Videourodynamics (VUDS) is the study of bladder and urethra function, using pressure studies in combination with cystourthrography. In a typical videourodynamic study, small catheters are placed into the bladder and rectum of the patient to measure pressures, and X-ray images of the lower urinary tract are obtained as the bladder is filled and during voiding. Typically, contrast material, which shows up well on an X-ray picture, is injected into the bladder for the purpose of the study. The information gleaned by the process, such as bladder shape and outline, reflux, lag time, presence of leakage, etc., is useful for the diagnosis of urinary tract dysfunction such as urinary incontinence, incomplete emptying or complete retention. However, the practical need in many cases for the use of contrast media, such as Iodine contrast materials, renders this method unpleasant for children. Further, the X-ray exposure associated with this method renders it relatively undesirable for use with children, and poses limitations on its use even with adults. Further limitations with X-ray VUDS include an inability to delineate pelvic floor muscle contraction, relaxation and straining manoeuvres during bladder filling and voiding. Also, valid VUDS values can be difficult to obtain in the face of massive reflux. Other imaging methods have been developed which can provide images of the lower urinary tract without the drawbacks associated with X-ray imaging; of interest in this regard is fast magnetic resonance imaging (MRI). However, MRI has its own associated hazards; objects may be attracted to the imaging magnet with sufficient force to injure the MRI machine and any intervening patients or personnel.

### SUMMARY OF THE INVENTION

The present invention includes a method for obtaining information about the function of the lower urinary tract of a patient.

The method comprises the steps of: (i) measuring the pressure within the lower urinary tract of the patient; and, simultaneously (ii) imaging the lower urinary tract and dynamic pelvic floor muscles with an MRI machine.

The present invention also includes a system for obtaining information about the function of the lower urinary tract of a patient, using an MRI machine. The system includes means for measuring the pressure within the lower urinary tract while it is being imaged by the MRI machine.

The method and system permit a physician to obtain images of and measurements of the pressure within the lower urinary tract of a patient, without the drawbacks associated with X-ray videourodynamics, namely, X-ray exposure and the need for the patient to be exposed to contrast media. The method and system can be used for the evaluation of patients with bladder, striated sphincter and bladder neck dysfunction or pelvic floor interaction on bladder function. The method also provides significant hereto unavailable advantages. Firstly, it provides the ability to determine the detrusor-pelvic floor synergy or dyssynergia during bladder rest, voiding, bladder outlet function or dysfunction or unstable bladder contraction. As well, it provides the ability to evaluate bladder neck function or dysfunction (detrusor-bladder neck synergy or dyssnergia, measurement of lag time, unstable bladder neck, internal sphincter-external urinary sphincter dyssnergia). Further, it provides the ability to accurately measure post voiding urine residue, and to diagnose spastic pelvic floor syndrome as a different entity with a different pathophysiology from an initially normal urinary tract anatomy and function.

Other advantages, features and characteristics of the present invention, as well as methods of operation and functions of the related elements of the structure, and the combination of parts and economies of manufacture, will become more apparent upon consideration of the following detailed description and the appended claims.

### DETAILED DESCRIPTION

In the method, the pressure within the lower urinary tract of a patient is measured, and, simultaneously, the lower urinary tract is imaged with an MRI machine. A suitable system for pressure measurement can include a silicon or silastic catheter coupled by a long (150 cm) plastic tube to a water filled transducer, which in turn is operatively coupled to a processor for pressure measurement. Using a tip catheter transducer may cause severe bladder injuries and is contraindicated in this procedure. The processor preferably takes the form of a urodynamic machine, such as the processor sold by Laborie Medical Technologies Inc. under the trade-mark DELPHIS. If a DELPHIS processor is used, a case of plastic or other MR friendly material is substituted for the standard metal case. As well, EMG leads of non-Ferroalloy, that is, of MR friendly metal, are substituted for the leads provided with the DELPHIS system.

The positioning of the catheter or catheters depends upon the nature of the study to be conducted. This is well-known to persons of ordinary skill in the science of urodynamics, and as such, is not disclosed herein in detail. However, it should be noted that the processor and transducer(s) should be maintained at a distance of at least 150 cm from the MRI machine, to minimize the potential for dangerous magnetic attraction. Where a urodynamic machine is used, it should be calibrated in the MRI room, in order to ameliorate the impact of the magnetic field produced by the MRI machine. Further, it should be noted that a steel infusion stand should not be used in the vicinity of the MRI machine; an infusion stand of wood or plastic is safe and appropriate. With the catheter or catheters suitably positioned, the patient is placed in the coil of the MRI machine, and the study is conducted. Typically, this will include measurements of pressure in the lower urinary tract, and imaging of the lower urinary tract, as the bladder is filled and during voiding.

The method surprisingly appears to be capable of providing the same information as that obtainable through X-ray videourodynamics; no change in the cystometry results appears to flow from the use of the method, and the introduction of urodynamic equipment into the MRI room is safe, not only for the machinery but also for the patients and the staff. In addition, the method can provide additional information on lag time measurement and pelvic floor activity, not only during micturition, but also with every unstable bladder contraction. It is also possible to differentiate 3 types of detrusor sphincter dyssnergia according to Blavis' classification. Yet further, delineation and movement of the urethra, bladder, uterus, vagina, rectum and small bowel can be shown in a reliable manner.

The following two examples will reinforce for the reader the advantage that can be derived by the method.

In the first example, five adult male rabbits were catheterized by a double lumen 7 Fr. urethral catheter under mild sedation by a single intramuscular injection of Acepromazine [0.5 mg/kg of body weight] approximately 2 hours before the procedure. A rectal line was also placed in an appropriate site for the measurement of intra-abdominal pressure. A urodynamic study was performed in an environmentally free, separate room, to provide baseline cystometric data. The animals were then transferred to the MRI room, with the same catheter, and placed in the coil apparatus. The levels of all transducers were adjusted to the level of the bladder, and all connecting tubes were held in a parallel and horizontal manner in order not to change the vesical pressure. The urodynamic machine was calibrated at the MRI room in order to decrease the magnetic field effect on the surroundings. Spinal MRI, MR Urogram (visualization of urinary system during study) as well as pelvic floor dynamic imaging were performed during the urodynamic study.

The cystomeric findings were compared, with and without MRI imaging, to find out any possible differences between the two studies in each animal and to rule out any negative effects on the study in the magnet room. There were no adverse effects on animals during the procedures. The urodynamic study results were similar in each animal with and without dynamic pelvic floor fast imaging. The whole bladder function (contraction, relaxation), bladder neck function, the lag time measurement, post voiding residue and striated sphincter as well as the entire urethral function during bladder rest, cough, and straining, was found to be visible and measurable in this procedure.

In the second example, twelve children (6 boys, 6 girls) with documented neuropathic bladder due to myelomeningocele (n=6) and dysfunctional voiding without any neurological deficit (n=6) were included in a study. The children had an average age of 6.5 years, ranging from 3-12 years. For each patient, a double lumen 7 Fr. urethral catheter with an in situ rectal line were connected to a DELPHIS urodynamic apparatus with a compact computer processing, and the catheters were connected by a 150 cm long plastic tube to the water transducer. An infusion pump set was used in 2 older children for bladder filling. Infusion saline was suspended from a wooden infusion stand. Three cycles of filling, straining and voiding were observed in each individual and all cystometric findings as well as pelvic floor dynamic studies were recorded on different computers with both machine's clocks synchronized. The studies show that tight bottom syndrome is visible with the tight pelvic floor and the striated sphincter dyssynergia with a decreased flow rate during maximum detrusor contraction and changes in bladder outline and shape. These are objective findings which have not been reportable previously. In a child with history of detrusore sphincter dyssynergia (DSD) an injection of Botulinum toxin into the striated sphincter confirmed a relaxed sphincter in conjunction with tight pelvic floor muscles. Most of the findings were not previously visible on X-ray video-urodynamics.

Whereas only a single preferred embodiment of the method and system have each herein been described, and whereas only two examples have been described, it will be readily understood by persons of ordinary skill in the art that various changes to the method and system may be made without departing from the spirit or scope of the invention. Accordingly, the invention is accordingly to be limited only by the claims appended hereto, purposively construed.

What is claimed is:

1. A method for obtaining information about the function of the lower urinary tract of a patient, the method comprising the steps of:
measuring the pressure within the lower urinary tract of the patient; and
simultaneously imaging the lower urinary tract and carrying out a dynamic pelvic floor study of the patient with an MRI machine.

2. A method according to claim 1, wherein the pressure within the lower urinary tract of the patient is measured, and the lower urinary tract is imaged, as the bladder of the patient is filled and during voiding.

3. A method according to claim 2, further comprising the step of:
measuring muscle activity in the lower urinary tract and pelvic floor muscles of the patient contemporaneously with measurement of the pressure and muscle activity in the lower urinary tract of the patient.

4. A method according to claim 3, wherein the pressure and muscle activity within the lower urinary tract of the patient is measured, and the lower urinary tract is imaged, as the bladder of the patient is filled and during voiding.

5. A method according to claim 1, wherein the pressure is measured using a catheter which extends into the patient's bladder through the patient's urethra.

6. A method according to claim 1, wherein the pressure is measured using a catheter which extends into the patient's bladder through the patient's urethra.

7. A method according to claim 6, wherein the catheter is a double lumen catheter.

8. A method according to claim 5, wherein the pressure is measured using a water-filled transducer coupled to the catheter by a plastic tube of about 150 cm in length.

9. A method according to claim 8, wherein the pressure is measured using a processor operatively coupled to the transducer.

10. A method according to claim 9, wherein the transducer and the processor are disposed at least 150 cm apart from the MRI machine in use.

11. A system, for use with an MRI machine, for obtaining information as to the function of the lower urinary tract of a patient, the system comprising means for measuring the pressure within the lower urinary tract of the patient while the lower urinary tract is imaged by the MRI machine.

12. A system according to claim 11, wherein the means for measuring pressure comprises a urethral catheter.

13. A system according to claim 12, wherein the catheter is constructed of silicon or silastic.

14. A system according to claim 13, wherein the catheter is a double lumen catheter.

15. A system according to claim 12, wherein the means for measuring pressure further comprises a water filled transducer coupled to the catheter by a plastic tube of about 150 cm in length.

16. A system according to claim 15, wherein the means for measuring pressure further comprises a processor operatively coupled to the transducer.

17. A system according to claim 16, wherein the transducer and the processor, in use, are disposed at least 150 cm apart from the MRI machine.

18. A system according to claim 14, wherein the means for measuring pressure further comprises a water filled transducer coupled to the catheter by a plastic tube of about 150 cm in length.

19. A system according to claim 18, wherein the means for measuring pressure further comprises a processor operatively coupled to the transducer.

20. A system according to claim 19, wherein the transducer and the processor, in use, are disposed at least 150 cm apart from the MRI machine.
